SEQUENCE LISTING (5 sequences)

SEQ ID NO: 1 - DNA, 993 bp, Homo sapiens
atgtggctcctggtcagtgtaattctaatctcacggatatcctctgttgggggagaagcaacattttgtgattttccaaaaataaaccatggaattctatatgatgaagaaaaatataagccattttcccaggttcctacaggggaagttttctattactcctgtgaatataattttgtgtctccttcaaaatcattttggactcgcataacatgcacagaagaaggatggtcaccaacaccaaagtgtctcagactgtgtttctttccttttgtggaaaatggtcattctgaatcttcaggacaaacacatctggaaggtgatactgtgcaaattatttgcaacacaggatacagacttcaaaacaatgagaacaacatttcatgtgtagaacggggctggtccacccctcccaaatgcaggtccactgacacttcctgtgtgaatccgcccacagtacaaaatgctcatatactgtcgagacagatgagtaaatatccatctggtgagagagtacgttatgaatgtaggagcccttatgaaatgtttggggatgaagaagtgatgtgtttaaatggaaactggacagaaccacctcaatgcaaagattctacgggaaaatgtgggccccctccacctattgacaatggggacattacttcattcccgttgtcagtatatgctccagcttcatcagttgagtaccaatgccagaacttgtatcaacttgagggtaacaagcgaataacatgtagaaatggacaatggtcagaaccaccaaaatgcttacatccgtgtgtaatatcccgagaaattatggaaaattataacatagcattaaggtggacagccaaacagaagctttatttgagaacaggtgaatcagctgaatttgtgtgtaaacggggatatcgtctttcatcacgttctcacacattgcgaacaacatgttgggatgggaaactggagtatccaacttgtgcaaaaagatag

SEQ ID NO: 2 - protein, 330 aa, Homo sapiens
MWLLVSVILISRISSVGGEATFCDFPKINHGILYDEEKYKPFSQVPTGEVFYYSCEYNFVSPSKSFWTRITCTEEGWSPTPKCLRLCFFPFVENGHSESSGQTHLEGDTVQIICNTGYRLQNNENNISCVERGWSTPPKCRSTDTSCVNPPTVQNAHILSRQMSKYPSGERVRYECRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYLRTGESAEFVCKRGYRLSSRSHTLRTTCWDGKLEYPTCAKR

SEQ ID NO: 3 - DNA, 61 bp, Homo sapiens
taaggtggacagccaaacagaagctttattcgagaacaggtgaatcagttgaatttgtgtg

SEQ ID NO: 4 - DNA, 61 bp, Homo sapiens
taaggtggacagccaaacagaagctttatttgagaacaggtgaatcagctgaatttgtgtg

SEQ ID NO: 5 - DNA, 26 bp, Artificial sequence
  chemically synthesized antisense polynucleotide
ttcagctgattcacctgttctcaaat

(19) United States
(12) Patent Application Publication
(10) Pub. No.: US 2009/0312394 A1
(43) Pub. Date: Dec. 17, 2009

(54) Protection against and treatment of age related macular degeneration

(75) Inventor: Anne Elizabeth Hughes, Belfast (GB)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: THE QUEEN'S UNIVERSITY OF BELFAST, Belfast (GB)

(21) Appl. No.: 12/308,342

(22) PCT Filed: Jun. 13, 2007
(86) PCT No.: PCT/GB2007/002207

§ 371 Date: Jan. 26, 2009

(30) Foreign Application Priority Data
Jun. 13, 2006 (GB) 0611606.5

(51) Int. Cl. A61K 31/7052; C07H 21/02; C40B 40/06; C40B 40/10; G01N 33/00; A61P 27/02

(52) U.S. Cl. 514/44 A; 536/24.5; 536/24.3; 506/16; 506/18; 436/94

(57) ABSTRACT

Methods and reagents in relation to the diagnosis, protection and treatment of Age Related Macular Degeneration (AMD). In particular, the methods and reagents in relation to RNA; determined to provide a strong protection to a subject against development of AMD.

### FIELD OF THE INVENTION

The present invention relates to the diagnosis, protection and treatment of age related macular degeneration (AMD). More particularly the invention relates to the identification of gene deletions which are common and are strongly protective against development of AMD and to inhibitors to silence these genes.

### BACKGROUND

Age-related macular degeneration (AMD) is the most common cause of visual impairment in the elderly population, with severe disease affecting nearly 10% of Caucasians over the age of 75 years. It is a complex disease in which genetic and environmental factors contribute to susceptibility. Complement factor H (CFH) has recently been identified as a major AMD susceptibility gene and the Y402H polymorphism has been proposed as the likely causative factor.

CFH and the closely related genes CFHL3, CFHL1, CFHL4, CFHL2 and CFHL5 (also known as CFHR 1-5) are arranged in tandem on chromosome 1q23 where they span 355 kb at the proximal end of the RCA gene cluster. The extremely high level of homology, particularly between the 3′ exons of CFH and CFHL1 (98%), and between exons of CFHL3 and CFHL4 (88-99%), suggests that they arose through genomic duplication. The gene products are involved in regulation of complement activity, a cascade implicated in formation of drusen which arise between Bruch's membrane and the retinal pigment epithelium in early AMD¹

### SUMMARY OF THE INVENTION

Despite evidence of the genetic components of AMD, specific genetic markers that can be used to test for disease susceptibility and/or which can be used as a means of preventing and/or treating AMD have not been identified.

According to a first aspect of the present invention there is provided a medicament for the prevention of and/or treatment for AMD, the medicament comprising at least one inhibitor to wholly or partly silence at least one of gene CFHL1 and/or gene CFHL3.

Reference sequence for CFHR1: NM_{—}002113.2.

mRNA for CFHR1 Nucleotide Sequence (993 nt):

(SEQ ID NO 1)

ATGTGGCTCCTGGTCAGTGTAATTCTAATCTCACGGATATCCTCTGTTGG
GGGAGAAGCAACATTTTGTGATTTTCCAAAAATAAACCATGGAATTCTAT
ATGATGAAGAAAAATATAAGCCATTTTCCCAGGTTCCTACAGGGGAAGTT
TTCTATTACTCCTGTGAATATAATTTTGTGTCTCCTTCAAAATCATTTTG
GACTCGCATAACATGCACAGAAGAAGGATGGTCACCAACACCAAAGTGTC
TCAGACTGTGTTTCTTTCCTTTTGTGGAAAATGGTCATTCTGAATCTTCA
GGACAAACACATCTGGAAGGTGATACTGTGCAAATTATTTGCAACACAGG
ATACAGACTTCAAAACAATGAGAACAACATTTCATGTGTAGAACGGGGCT
GGTCCACCCCTCCCAAATGCAGGTCCACTGACACTTCCTGTGTGAATCCG
CCCACAGTACAAAATGCTCATATACTGTCGAGACAGATGAGTAAATATCC
ATCTGGTGAGAGAGTACGTTATGAATGTAGGAGCCCTTATGAAATGTTTG
GGGATGAAGAAGTGATGTGTTTAAATGGAAACTGGACAGAACCACCTCAA
TGCAAAGATTCTACGGGAAAATGTGGGCCCCCTCCACCTATTGACAATGG
GGACATTACTTCATTCCCGTTGTCAGTATATGCTCCAGCTTCATCAGTTG
AGTACCAATGCCAGAACTTGTATCAACTTGAGGGTAACAAGCGAATAACA
TGTAGAAATGGACAATGGTCAGAACCACCAAAATGCTTACATCCGTGTGT
AATATCCCGAGAAATTATGGAAAATTATAACATAGCATTAAGGTGGACAG
CCAAACAGAAGCTTTATTTGAGAACAGGTGAATCAGCTGAATTTGTGTGT
AAACGGGGATATCGTCTTTCATCACGTTCTCACACATTGCGAACAACATG
TTGGGATGGGAAACTGGAGTATCCAACTTGTGCAAAAAGATAG
Translation (330 aa):

(SEQ ID NO: 2)

MWLLVSVILISRISSVGGEATFCDFPKINHGILYDEEKYKPFSQVPTGEV
FYYSCEYNFVSPSKSFWTRITCTEEGWSPTPKCLRLCFFPFVENGHSESS
GQTHLEGDTVQIICNTGYRLQNNENNISCVERGWSTPPKCRSTDTSCVNP
PTVQNAHILSRQMSKYPSGERVRYECRSPYEMFGDEEVMCLNGNWTEPPQ
CKDSTGKCGPPPPIDNGDITSFPLSVYAPASSVEYQCQNLYQLEGNKRIT
CRNGQWSEPPKCLHPCVISREIMENYNIALRWTAKQKLYLRTGESAEFVC
KRGYRLSSRSHTLRTTCWDGKLEYPTCAKR

As used herein, the term “gene” refers to a nucleic acid (e.g., DNA) sequence that comprises coding sequences necessary for the production of a polypeptide or precursor. The polypeptide can be encoded by a full length coding sequence or by any portion of the coding sequence so long as the desired activity or functional properties (e.g., enzymatic activity, ligand binding, signal transduction, etc.) of the full-length or fragment are retained. The term “gene” encompasses both cDNA and genomic forms of a gene. A genomic form or clone of a gene contains the coding region interrupted with non-coding sequences termed “intervening regions” or “intervening sequences.”

As used herein, the term “gene silencing” refers to a phenomenon whereby a function of a gene is completely or partially inhibited. Throughout the specification, the terms “silencing,” “inhibition,” “quelling,” “knockout” and “suppression,” when used with reference to reduction of gene transcription, protein expression or function of expressed protein, are used interchangeably.

Prevention of AMD, is considered to be reducing the risk of a subject in developing in AMD at a later timepoint. Treatment of AMD is slowing of the progression of AMD and/or reversal of symptoms of AMD in a subject.

The at least one inhibitor of the present invention can comprise RNAi.

### RNAi

RNA interference (RNAi) or posttranscriptional gene silencing (PTGS) is a process whereby double-stranded RNA induces potent and specific gene silencing. RNAi is mediated by RNA-induced silencing complex (RISC), a sequence-specific, multicomponent nuclease that destroys messenger RNAs homologous to the silencing trigger. RISC is known to contain short RNAs (approximately 22 nucleotides) derived from the double-stranded RNA trigger.

In one aspect, the invention provides methods of employing an RNAi agent to modulate expression, preferably reducing expression of a target gene, CFHL1 or CFHL3, in a mammalian, preferably human host. By reducing expression is meant that the level of expression of a target gene or coding sequence is reduced or inhibited by at least about 2-fold, usually by at least about 5-fold, e.g., 10-fold, 15-fold, 20-fold, 50-fold, 100-fold or more, as compared to a control. In certain embodiments, the expression of the target gene is reduced to such an extent that expression of the CFHL1 or CFHL3 gene/coding sequence is effectively inhibited. By modulating expression of a target gene is meant altering, e.g., reducing, translation of a coding sequence, e.g., genomic DNA, mRNA etc., into a polypeptide, e.g., protein, product.

The RNAi agents that may be employed in preferred embodiments of the invention are small ribonucleic acid molecules (also referred to herein as interfering ribonucleic acids), that are present in duplex structures, e.g., two distinct oligoribonucleotides hybridized to each other or a single ribooligonucleotide that assumes a small hairpin formation to produce a duplex structure. Preferred oligoribonucleotides are ribonucleic acids of not greater than 100 nt in length, typically not greater than 75 nt in length. Where the RNA agent is an siRNA, the length of the duplex structure typically ranges from about 15 to 30 bp, usually from about 20 and 29 bps, most preferably 21 bp. Where the RNA agent is a duplex structure of a single ribonucleic acid that is present in a hairpin formation, i.e., a shRNA, the length of the hybridized portion of the hairpin is typically the same as that provided above for the siRNA type of agent or longer by 4-8 nucleotides.

In certain embodiments, instead of the RNAi agent being an interfering ribonucleic acid, e.g., an siRNA or shRNA as described above, the RNAi agent may encode an interfering ribonucleic acid. In these embodiments, the RNAi agent is typically DNA that encodes the interfering ribonucleic acid. The DNA may be present in a vector.

The RNAi agent can be administered to the host using any suitable protocol known in the art. For example, the nucleic acids may be introduced into tissues or host cells by viral infection, microinjection, fusion of vesicles, particle bombardment, or hydrodynamic nucleic acid administration.

DNA directed RNA interference (ddRNAi) is an RNAi technique which may be used in the methods of the invention. ddRNAi is described in U.S. Pat. No. 6,573,099 and GB 2353282. ddRNAi is a method to trigger RNAi which involves the introduction of a DNA construct into a cell to trigger the production of double stranded (dsRNA), which is then cleaved into small interfering RNA (siRNA) as part of the RNAi process. ddRNAi expression vectors generally employ RNA polymerase III promoters (e.g. U6 or H1) for the expression of siRNA target sequences transfected in mammalian cells. siRNA target sequences generated from a ddRNAi expression cassette system can be directly cloned into a vector that does not contain a U6 promoter. Alternatively short single stranded DNA oligos containing the hairpin siRNA target sequence can be annealed and cloned into a vector downsteam of the pol III promoter. The primary advantages of ddRNAi expression vectors is that they allow for long term interference effects and minimise the natural interferon response in cells.

An example of suitable methodology in relation to the design and use of SiRNA, as could be applied by a person of skill in the art, following the novel and inventive determination by the inventors of the genetic components associated to AMD is provided by Soutschek J, Akinc A, Bramlage B. Charisse K, Constien R, Donoghue M, Elbashir S, Geick A, Hadwiger P, Harborth J, John M, Kesavan V, Lavine G, Pandey R K, Racie T, Rajeev K G, Rohl I, Toudjarska I, Wang G, Wuschko S, Bumcrot D, Koteliansky V, Limmer S, Manoharan M, Vomlocher H P (2004) Therapeutic silencing of an endogenous gene by systemic administration of modified siRNAs. Nature 432(7014):173-178. This paper discusses the screening of 84 siRNAs targeting mouse and human apoB in HepG2 liver cells for their ability to reduce apoB mRNA and protein levels. Two of the most potent siRNAs were chemically modified and conjugated to cholesterol, which was shown to confer improved pharmacological properties on siRNAs in vitro and in vivo. These siRNAs were shown to reduce apoB mRNA in liver and jejunum (the primary sites of apoB expression), decrease plasma levels of apoB protein, and reduce total cholesterol after intravenous injection in mice. Cleavage of apoB mRNA was shown to occur specifically at the site predicted by current models of RNAi, 10 nt downstream of the 5′ end of the siRNA antisense strand.

### Anti-Sense RNA

CFHL1 or CFHL3 inhibitors for use in the invention may be anti-sense molecules or nucleic acid constructs that express such anti-sense molecules as RNA. The antisense molecules may be natural or synthetic. Synthetic antisense molecules may have chemical modifications from native nucleic acids. The antisense sequence is complementary to the mRNA of the targeted CFHL1 or CFHL3 gene, and inhibits expression of the targeted gene products. Antisense molecules inhibit gene expression through various mechanisms, e.g. by reducing the amount of mRNA available for translation, through activation of RNAse H, or steric hindrance. One or a combination of antisense molecules may be administered, where a combination may comprise multiple different sequences.

Antisense molecules may be produced by expression of all or a part of the CFHL1 gene or CFHL3 gene sequence in an appropriate vector, where the transcriptional initiation is oriented such that an antisense strand is produced as an RNA molecule. Alternatively, the antisense molecule may be a synthetic oligonucleotide. Antisense oligonucleotides will generally be at least about 7, usually at least about 12, more usually at least about 16 nucleotides in length, and usually not more than about 50, preferably not more than about 35 nucleotides in length.

A specific region or regions of the endogenous CFHL1 or CFHL3 sense strand mRNA sequence can be chosen to be complemented by the antisense sequence.

In particular embodiments, the at least one antisense sequence can be complementary to a nucleotide sequence comprising at least 90%, at least 95%, at least 99% and preferably at least 100% sequence identity to:

(SEQ ID NO: 3)

CFH: taaggtggacagccaaacagaagctttattcgagaacaggtgaat
cagttgaatttgtgtg
or

(SEQ ID NO: 4)

CFHR1: taaggtggacagccaaacagaagctttatttgagaacaggtga
atcagctgaatttgtgtg.

### (Differences in the Nucleotide Bases are Shown by Underlining)

Suitably, one embodiment of an inhibitor can be an antisense sequence which would be complementary to CFHR1 such as a nucleotide sequence which comprises or is ttcaGctgattcacctgttctcAaat (SEQ ID NO: 5) or a polynucleotide sequence which has at least 90%, at least 95%, at least 99%, at least 100% sequence identity to said sequence.

Selection of a specific sequence for the oligonucleotide can be determined through the use of an empirical method, where several candidate sequences are assayed for inhibition of expression of the target gene in an in vitro or animal model. A combination of sequences may also be used, where several regions of the mRNA sequence are selected for antisense complementation.

Antisense oligonucleotides may be chemically synthesized by methods known in the art (see Wagner et al. (1993), supra, and Milligan et al., supra.) Preferred oligonucleotides are chemically modified from the native phosphodiester structure, in order to increase their intracellular stability and binding affinity. A number of such modifications have been described in the literature, which alter the chemistry of the backbone, sugars or heterocyclic bases. Among useful changes in the backbone chemistry are phosphorodiamidate linkages, methylphosphonates phosphorothioates; phosphorodithioates, where both of the non-bridging oxygens are substituted with sulfur; phosphoroamidites; alkyl phosphotriesters and boranophosphates. Achiral phosphate derivatives include 3′-O-5′-S-phosphorothioate, 3′-S-5-O-phosphorothloate, 3′-CH2-5′-O-phosphonate and 3′-NH-5-O-phosphoroamidate. Peptide nucleic acids may replace the entire ribose phosphodiester backbone with a peptide linkage. Sugar modifications may also be used to enhance stability and affinity.

According to a second aspect of the present invention there is provided the use of at least one inhibitor to wholly or partly silence at least one of gene CFHL1 and/or gene CFHL3 in medicine.

According to a third aspect of the present invention there is provided the use of at least one inhibitor to wholly or partly silence at least one of gene CFHL1 and/or gene CFHL3 in the preparation of a medicament for the treatment of AMD.

According to a fourth aspect of the present invention there is provided a method of treating AMD comprising the step of providing at least one inhibitor to wholly or partly silence at least one of gene CFHL1 and/or gene CFHL3 to a patient in need thereof.

In particular embodiments of the second, third and fourth aspects of the invention, the at least one inhibitor can be an antisense molecule or RNAi as discussed herein.

In particular embodiments of the first, second, third and fourth aspects of the invention the at least one inhibitor to wholly or partly silence at least one of gene CFHL1 and/or gene CFHL3 can be provided in combination with another treatment.

In particular embodiments of the first, second, third and fourth aspects of the invention the at least one inhibitor to wholly or partly silence at least one of gene CFHL1 and or gene CFHL3 can be provided in combination with anti-VEGF treatment.

Anti-VEGF treatments target VEGF (Vascular endothelial growth factor), a protein that helps the formation of new blood vessels. In AMD it has been suggested that new blood vessels are unstable and tend to leak fluid and blood under the retina. This is thought to result in scarring which causes irreversible sight loss. In the case of AMD, it is considered that anti-VEGF treatments inhibit the growth of new blood vessels, and thus minimise the risk of scarring.

Anti-VEGF treatment includes, for example, Macugen, Avastin, Lucentis or the like.

Suitably, the at least one inhibitor to wholly or partly silence at least one of gene CFHL1 and/or gene CFHL3 can be provided in combination with a drug which minimises the likelihood of a patient smoking, for example, Champix, bupropion or the like.

### Treatment

Treatment” includes any regime that can benefit a human or non-human animal. The treatment may be in respect of an existing AMD condition or may be prophylactic (preventative treatment). Treatment may include curative, alleviation or prophylactic effects of AMD.

### Administration

CFHL1 and CFHL3 inhibitors of and for use in the present invention may be administered in any suitable way. Moreover they can be used in combination or in combination with other therapy. In such embodiments, the inhibitors or compositions of the invention may be administered simultaneously, separately or sequentially with another chemotherapeutic agent.

Where administered separately or sequentially, they may be administered within any suitable time period e.g. within 1, 2, 3, 6, 12, 24, 48 or 72 hours of each other. In preferred embodiments, they are administered within 6, preferably within 2, more preferably within 1, most preferably within 20 minutes of each other.

In a preferred embodiment, the inhibitors and/or compositions of the invention are administered as a pharmaceutical composition, which will generally comprise a suitable pharmaceutical excipient, diluent or carrier selected dependent on the intended route of administration.

The inhibitors and/or compositions of the invention may be administered to a patient in need of treatment via any suitable route.

Targeting therapies may be used to deliver the active agents more specifically to certain types of cell, by the use of targeting systems such as antibody or cell specific ligands. Targeting may be desirable for a variety of reasons, for example if the agent is unacceptably toxic, or if it would otherwise require too high a dosage, or if it would not otherwise be able to enter the target cells.

For intravenous, injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringers Injection, Lactated Ringer's Injection. Preservatives, stabilisers, buffers, antioxidants and/or other additives may be included, as required. Accordingly, the present invention includes a pharmaceutical composition comprising a medicament of the first aspect of the invention.

Pharmaceutical compositions for oral administration may be in tablet, capsule, powder or liquid form. A tablet may comprise a solid carrier such as gelatin or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

The inhibitors and/or compositions of the invention may also be administered via microspheres, liposomes, other microparticulate delivery systems or sustained release formulations placed in certain tissues including blood. Suitable examples of sustained release carriers include semipermeable polymer matrices in the form of shared articles, e.g. suppositories or microcapsules. Implantable or microcapsular sustained release matrices include polylactides (U.S. Pat. No. 3,773,919; EP-A-0058481) copolymers of L-glutamic acid and gamma ethyl-L-glutamate (Sidman et al, Biopolymers 22(1): 547-556, 1985), poly(2-hydroxyethyl-methacrylate) or ethylene vinyl acetate (Langer et al, J. Biomed. Mater. Res. 15: 167-277, 1981, and Langer, Chem. Tech. 12:98-105, 1982). Liposomes containing the polypeptides are prepared by well-known methods: DE 3,218,121A; Epstein et al, PNAS USA, 82: 3688-3692, 1985; Hwang et al, PNAS USA, 77: 4030-4034, 1980; EP-A-052522; E-A-0036676; EP-A-0088046; EP-A-0143949; EP-A-0142541; JP-A-83-11808; U.S. Pat. Nos. 4,485,045 and 4,544,545. Ordinarily, the liposomes are of the small (about 200-800 Angstroms) unilamellar type in which the lipid content is greater than about 30 mol. % cholesterol, the selected proportion being adjusted for the optimal rate of the polypeptide leakage.

Examples of the techniques and protocols mentioned above and other techniques and protocols which may be used in accordance with the invention can be found in Remington's Pharmaceutical Sciences, 16th edition, Oslo, A. (ed), 1980.

### Pharmaceutical Compositions

Pharmaceutical compositions according to the present invention, and for use in accordance with the present invention may comprise, in addition to active ingredients, a pharmaceutically acceptable excipient, carrier, buffer stabiliser or other materials well known to those skilled in the art. Such materials should be non-toxic and should not interfere with the efficacy of the active ingredient. The precise nature of the carrier or other material will depend on the route of administration, which may be oral, or by injection, e.g. intravenous.

The formulation may be a liquid, for example, a physiologic salt solution containing non-phosphate buffer at pH 6.8-7.6, or a lyophilised powder.

### Dose

The inhibitors or compositions of the invention are preferably administered to an individual in a “therapeutically effective amount”, this being sufficient to show benefit to the individual. The actual amount administered, and rate and time-course of administration, will depend on the nature and severity of what is being treated. Prescription of treatment, e.g. decisions on dosage etc, is ultimately within the responsibility and at the discretion of general practitioners and other medical doctors, and typically takes account of the disorder to be treated, the condition of the individual patient, the site of delivery, the method of administration and other factors known to practitioners.

The inventor has determined novel polymorphisms of genes which are associated to AMD and accordingly a fifth aspect of the present invention is at least one probe comprising an isolated polynucleotide sequence that comprises one or more polymorphisms selected from the list:

| SNP Number | SNP Name |
|---|---|
| 1 | rs1292487 |
| 2 | rs512900 |
| 3 | rs7524776 |
| 4 | rs529825 |
| 5 | rs800292 |
| 6 | rs1329424 |
| 7 | rs1061147 |
| 8 | rs1061170 |
| 9 | rs10801555 |
| 10 | rs2019727 |
| 11 | rs2019724 |
| 12 | rs203685 |
| 13 | rs1831281 |
| 14 | rs2274700 |
| 15 | rs6677604 |
| 16 | rs3753396 |
| 17 | rs419137 |
| 18 | rs2284664 |
| 19 | rs1065489 |
| 20 | rs10801560 |
| 21 | rs460897 |
| 22 | rs432007 |
| 23 | rs438781 |
| 24 | rs408519 |
| 25 | rs6428372 |
| 26 | rs10922147 |
| 27 | rs1971579 |
| 28 | rs4085749 |
| 29 | rs10922152 |
| 30 | rs5998 |

Suitably, at least one probe comprising an isolated polynucleotide sequence that comprises one or more polymorphisms selected from the list
5 rs800292
8 rs1061170
15 rs6677604
16 rs3753396
17 rs419137
18 rs2284664

As used herein the term “isolated polynucleotide sequence that comprises one or more polymorphism” is one that contains an SNP of the present invention and is separated from other nucleic acid present in the natural source of the nucleic acid.

Isolated polynucleotide sequences can be in the form of RNA, such as mRNA, or in the form of DNA, including genomic NDA or cDNA. Alternatively, the polynucleotide sequences can be obtained by chemical synthesis methods. The polynucleotide sequences can be double stranded or single stranded.

The contribution or association of particular SNPs with disease phenotypes of AMD enables SNPs to be used to develop superior diagnostic tests capable of identifying individuals who express detectable traits and which places them at increased/decreased risk of developing AMD at a subsequent time. Diagnosis may be based on a single SNP or a group of SNPs. Combined detection of a plurality of SNPs typically increase the probability of accurate diagnosis.

The presence or absence of particular SNPs/haplotypes to diagnose, predict susceptibility to or monitor a subject in relation to AMD can be determined using methods as known to a person of skill in the art, including, for example, enzymatic amplification of nucleic acid from a sample from the subject followed by DNA sequence analysis, primer extension methodology or mass spectrometry.

Association studies in patients with disease and unaffected controls can indicate which polymorphisms and/or haplotypes confer protection or increased risk of disease.

As will be appreciated by those of skill in the art, where particular SNPs have been illustrated in the present application, alternative SNPs can be utilised to define the haplotype structure of each genetic locus where the alternative SNPs are in perfect linkage disequilibrium with those that are defined.

The international HapMap Project and other genomic sequencing efforts have elucidated the pattern of polymorphisms on common haplotypes. Often different combinations of polymorphic variants can be typed to gain full haplotypic information in an individual. These combinations of markers are known as haplotype tagging polymorphisms.

According to a sixth aspect of the present invention, there is provided a diagnostic kit for the diagnosis and/or monitoring of age related macular degeneration in a subject, said kit comprising: a detection reagent with binding specificity for a polynucleotide sequence comprising one or more polymorphisms selected from the list:

| SNP Number | SNP Name |
|---|---|
| 1 | rs1292487 |
| 2 | rs512900 |
| 3 | rs7524776 |
| 4 | rs529825 |
| 5 | rs800292 |
| 6 | rs1329424 |
| 7 | rs1061147 |
| 8 | rs1061170 |
| 9 | rs10801555 |
| 10 | rs2019727 |
| 11 | rs2019724 |
| 12 | rs203685 |
| 13 | rs1831281 |
| 14 | rs2274700 |
| 15 | rs6677604 |
| 16 | rs3753396 |
| 17 | rs419137 |
| 18 | rs2284664 |
| 19 | rs1065489 |
| 20 | rs10801560 |
| 21 | rs460897 |
| 22 | rs432007 |
| 23 | rs438781 |
| 24 | rs408519 |
| 25 | rs6428372 |
| 26 | rs10922147 |
| 27 | rs1971579 |
| 28 | rs4085749 |
| 29 | rs10922152 |
| 30 | rs5998 |

or to a molecule encoded by a polynucleotide sequence that comprises one or more polymorphisms selected from the list:

| SNP Number | SNP Name |
|---|---|
| 1 | rs1292487 |
| 2 | rs512900 |
| 3 | rs7524776 |
| 4 | rs529825 |
| 5 | rs800292 |
| 6 | rs1329424 |
| 7 | rs1061147 |
| 8 | rs1061170 |
| 9 | rs10801555 |
| 10 | rs2019727 |
| 11 | rs2019724 |
| 12 | rs203685 |
| 13 | rs1831281 |
| 14 | rs2274700 |
| 15 | rs6677604 |
| 16 | rs3753396 |
| 17 | rs419137 |
| 18 | rs2284664 |
| 19 | rs1065489 |
| 20 | rs10801560 |
| 21 | rs460897 |
| 22 | rs432007 |
| 23 | rs438781 |
| 24 | rs408519 |
| 25 | rs6428372 |
| 26 | rs10922147 |
| 27 | rs1971579 |
| 28 | rs4085749 |
| 29 | rs10922152 |
| 30 | rs5998 |

In certain embodiments said kit comprises at least two, at least three, at least four, at least five, at least six, at least ten, at least fifteen detection reagents with binding specificity to a polynucleotide sequence that comprises one or more polymorphisms selected from the list:

| SNP Number | SNP Name |
|---|---|
| 1 | rs1292487 |
| 2 | rs512900 |
| 3 | rs7524776 |
| 4 | rs529825 |
| 5 | rs800292 |
| 6 | rs1329424 |
| 7 | rs1061147 |
| 8 | rs1061170 |
| 9 | rs10801555 |
| 10 | rs2019727 |
| 11 | rs2019724 |
| 12 | rs203685 |
| 13 | rs1831281 |
| 14 | rs2274700 |
| 15 | rs6677604 |
| 16 | rs3753396 |
| 17 | rs419137 |
| 18 | rs2284664 |
| 19 | rs1065489 |
| 20 | rs10801560 |
| 21 | rs460897 |
| 22 | rs432007 |
| 23 | rs438781 |
| 24 | rs408519 |
| 25 | rs6428372 |
| 26 | rs10922147 |
| 27 | rs1971579 |
| 28 | rs4085749 |
| 29 | rs10922152 |
| 30 | rs5998 |

or to a polypeptide encoded by at least one of said polynucleotide sequences.

Suitably at least one detection reagent has binding specificity to a polynucleotide sequence that comprises one or more polymorphisms selected from the list:
5 rs800292
8 rs1061170
15 rs6677604
16 rs3753396
17 rs419137
18 rs2284664

Suitably the detection reagent can be a nucleotide sequence which is complementary to a polynucleotide sequence comprising any of the SNPs numbered 1 to 30 identified in the present application or to alternative SNPs in perfect linkage disequilibrium with those that are defined which define the haplotype structure of the genetic markers.

By complementary it is meant the detection reagent, when a nucleotide sequence, will hybridise to a polynucleotide sequence comprising any of the SNPs numbered 1 to 30 under at least stringent conditions.

As will be appreciated, where reference has been made to specific SNPS, as nucleic acid can be a double stranded molecule, reference to an SNP on one strand will in turn refer to a corresponding position on the complementry strand. Oligonucleotide probes or primers can be designed to hybridise to either strand.

In certain embodiments, the detection reagent is labelled with a reporter.

In certain embodiments, the reporter is fluorescent.

In certain embodiments the detection reagent is bound to a solid support.

In particular embodiments the detection reagent is bound to a solid substrate, including, paper, nylon, a filter or membrane, a chip, a glass slide as an array of distinct molecules. In certain embodiments the detection reagent is synthesised on the solid support. Arrays can be provided and used according to the methods disclosed in U.S. Pat. No. 5,837,832 and PCT application WO 95/1995.

In certain embodiments, the detection reagent is an array of said polynucleotide sequences, wherein said polynucleotide sequences are immobilized on a computer chip and hybridization of a nucleic acid molecule from a sample to the array can be detected using computerized technology.

Accordingly, a seventh aspect of the invention provides at least one array comprising at least two polynucleotide sequences capable of hybridizing to at least two genetic markers selected from polynucleotide sequence that comprise one or more polymorphisms selected from the list:

| SNP Number | SNP Name |
|---|---|
| 1 | rs1292487 |
| 2 | rs512900 |
| 3 | rs7524776 |
| 4 | rs529825 |
| 5 | rs800292 |
| 6 | rs1329424 |
| 7 | rs1061147 |
| 8 | rs1061170 |
| 9 | rs10801555 |
| 10 | rs2019727 |
| 11 | rs2019724 |
| 12 | rs203685 |
| 13 | rs1831281 |
| 14 | rs2274700 |
| 15 | rs6677604 |
| 16 | rs3753396 |
| 17 | rs419137 |
| 18 | rs2284664 |
| 19 | rs1065489 |
| 20 | rs10801560 |
| 21 | rs460897 |
| 22 | rs432007 |
| 23 | rs438781 |
| 24 | rs408519 |
| 25 | rs6428372 |
| 26 | rs10922147 |
| 27 | rs1971579 |
| 28 | rs4085749 |
| 29 | rs10922152 |
| 30 | rs5998 |

Suitably the array comprises at least two polynucleotide sequences capable of hybridizing to at least two genetic markers selected from polynucleotide sequence that comprise one or more polymorphisms selected from the list:
5 rs800292
8 rs1061170
15 rs6677604
16 rs3753396
17 rs419137
18 rs2284664

Said array can be used for diagnosing age-related macular degeneration by determining the genetic profile of a biological sample from a subject to determine the presence or absence of genetic markers for diagnosing age-related macular disease or monitoring the progression of age-related macular disease.

In particular embodiments, at least one array comprises three or more, for example four polynucleotide sequences, five polynucleotide sequences, six polynucleotide sequences, ten polynucleotide sequences, fifteen polynucleotide sequences capable of hybridizing to a genetic markers selected form polynucleotide sequence that comprise one or more polymorphisms selected from the list:

| SNP Number | SNP Name |
|---|---|
| 1 | rs1292487 |
| 2 | rs512900 |
| 3 | rs7524776 |
| 4 | rs529825 |
| 5 | rs800292 |
| 6 | rs1329424 |
| 7 | rs1061147 |
| 8 | rs1061170 |
| 9 | rs10801555 |
| 10 | rs2019727 |
| 11 | rs2019724 |
| 12 | rs203685 |
| 13 | rs1831281 |
| 14 | rs2274700 |
| 15 | rs6677604 |
| 16 | rs3753396 |
| 17 | rs419137 |
| 18 | rs2284664 |
| 19 | rs1065489 |
| 20 | rs10801560 |
| 21 | rs460897 |
| 22 | rs432007 |
| 23 | rs438781 |
| 24 | rs408519 |
| 25 | rs6428372 |
| 26 | rs10922147 |
| 27 | rs1971579 |
| 28 | rs4085749 |
| 29 | rs10922152 |
| 30 | rs5998 |

Hybridization to the array may be performed under conditions selected to provide a suitable degree of stringency. The skilled person is well aware of techniques for varying hybridization conditions in order to select the most appropriate degree of stringency for a particular sample. For example, using a non-stringent wash buffer and a stringent wash buffer a person of ordinary skill in the art can alter the number of respective washes (typically 0-20), the wash temperature (typically 15-50° C.) and hybridization temperature (typically 15-50° C.) to achieve optimal hybridization. Methods of optimizing hybridization conditions are well known to those of skill in the art (see, e.g., LABORATORY TECHNIQUES IN BIOCHEMISTRY AND MOLECULAR BIOLOGY, Vol. 24: Hybridization With Nucleic Acid Probes, P. Tijssen, ed. Elsevier, N.Y., (1993)). One of ordinary skill in the art may adjust hybridization factors to provide optimum hybridization and signal production for a given hybridization procedure and to provide the required resolution among different genes or genomic locations.

Hybridization or binding of transcripts within the biological sample with complementary sequences on the array under stringent conditions can then detected. Hybridisation under stringent conditions is intended to describe conditions under which nucleotide sequences of at least 60%, at least 70%, at least 80%, at least 90%, at least 95% or more homology to each other remain hybridized to each other. Such stringent conditions are well known to those in the art, for example Current Protocols in Molecular Biology, John Wiley & Sons, N.Y. (1989). “Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

As herein defined, “Stringent conditions”, may be identified by those that: (1) employ low ionic strength and high temperature for washing, for example 0.015 M sodium chloride/0.0015 M sodium citrate/0.1% sodium dodecyl sulfate at 50° C.; (2) employing during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employing 50% formamide, 5*SSC (0.75 M NaCl, 0.075 M sodium citrate), 50 mM sodium phosphate (pH 6.8), 0.1% sodium pyrophosphate, 5*Denhardt's solution, sonicated salmon sperm DNA (50 [mu]g/ml), 0.1% SDS, and 10% dextran sulfate at 42° C., with washes at 42° C. in 0.2*SSC (sodium chloride/sodium citrate) and 50% formamide at 55° C., followed by a high-stringency wash consisting of 0.1*SSC containing EDTA at 55° C.

“Moderately stringent conditions” may be identified as described by Sambrook et al., Molecular Cloning: A Laboratory Manual, New York: Cold Spring Harbor Press, 1989, and include the use of washing solution and hybridization conditions (e.g., temperature, ionic strength and % SDS) less stringent that those described above. An example of moderately stringent conditions is overnight incubation at 37° C. in a solution comprising: 20% formamide, 5*SSC (150 mM NaCl, 15 mM trisodium citrate), 50 mM sodium phosphate (pH 7.6), 5×Denhardt's solution, 10% dextran sulfate, and 20 mg/mL denatured sheared salmon sperm DNA, followed by washing the filters in 1*SSC at about 37-50° C. The skilled artisan will recognize how to adjust the temperature, ionic strength, etc. as necessary to accommodate factors such as probe length and the like. Highly stringent conditions may be based on the above, but with followed by washing the filters in 2*SSC at about 50° C. Very highly stringent followed by washing the filters in 6*SSC at about 65° C.

The nucleic acid sequences used in an array may be any type of nucleic acid or nucleic acid analog, including without limitation, RNA, DNA, peptide nucleic acids, or mixtures and/or fragments thereof. As used herein the term “fragment” refers to a nucleotide sequence that is a part of a sequence such as those provided herein that retains sufficient nucleotide sequence to permit the fragment to maintain specificity and selectivity to the whole sequence from which it is derived.

In particular embodiments, where large amounts of DNA are available, genomic DNA may be used directly. Alternatively, the region of interest can be cloned into a suitable vector and grown in sufficient quantity for analysis. The nucleotide sequence may be amplified by conventional techniques, such as the polymerase chain reaction (PCR) (Saiki, et al. (1985) Science 239:487). Primers may be used to amplify sequences encoding the polypeptide of interest. Optionally, a detectable label, for example a fluorochrome, biotin or a radioactive label may be used in such an amplification reaction. The label may be conjugated to one or both of the primers. Alternatively, the pool of nucleotides used in the amplification is labelled, so as to incorporate the label into the amplification product.

The sample nucleic acid, e.g. amplified or cloned may be analysed using any suitable method known in the art. For example, the nucleic acid may be sequenced by dideoxy or other methods, and the sequence of bases compared to the deleted sequence. Hybridization with the variant sequence may also be used to determine its presence, by Southern blots, dot blots, etc. The hybridization pattern of a control and variant sequence to an array of oligonucleotide probes immobilized on a solid support, as described in WO95/35505, may be used as a means of detecting the presence or absence of a sequence.

Alternatively, using standard techniques in the art, the presence of nucleic acids encoding the polypeptide or indeed an antibody specific to said polypeptide may be used. Further, the presence of antibodies specific to said polypeptides may be used to determine the presence of an immune response to said polypeptide.

It is well understood by those skilled in the art that cellular DNA in the form of genes is transcribed into RNA; coding RNA is translated into proteins; and RNA is optionally reverse-transcribed into cDNA.

The presence of particular genetic markers can be determined by detecting polypeptides encoded by a polynucleotide sequence that comprises one or more polymorphisms selected from the list:

| SNP Number | SNP Name |
|---|---|
| 1 | rs1292487 |
| 2 | rs512900 |
| 3 | rs7524776 |
| 4 | rs529825 |
| 5 | rs800292 |
| 6 | rs1329424 |
| 7 | rs1061147 |
| 8 | rs1061170 |
| 9 | rs10801555 |
| 10 | rs2019727 |
| 11 | rs2019724 |
| 12 | rs203685 |
| 13 | rs1831281 |
| 14 | rs2274700 |
| 15 | rs6677604 |
| 16 | rs3753396 |
| 17 | rs419137 |
| 18 | rs2284664 |
| 19 | rs1065489 |
| 20 | rs10801560 |
| 21 | rs460897 |
| 22 | rs432007 |
| 23 | rs438781 |
| 24 | rs408519 |
| 25 | rs6428372 |
| 26 | rs10922147 |
| 27 | rs1971579 |
| 28 | rs4085749 |
| 29 | rs10922152 |
| 30 | rs5998 |

or an immune response thereto using any means known in the art.

Suitably such means included, for example, an ELISA assay or RIA.

In one embodiment, the presence of a polypeptide in the sample can determined; alternatively or additionally the presence of an antibody specific to said polypeptide can be determined; alternatively or additionally the presence of a polynucleotide sequence encoding said antibody or said polypeptide is determined.

Accordingly an even further aspect of the present invention provides a polypeptide array, wherein said polypeptide array is comprised of
polypeptides encoded by any one polynucleotide sequence that comprises one or more polymorphisms selected from the list:

| SNP Number | SNP Name |
|---|---|
| 1 | rs1292487 |
| 2 | rs512900 |
| 3 | rs7524776 |
| 4 | rs529825 |
| 5 | rs800292 |
| 6 | rs1329424 |
| 7 | rs1061147 |
| 8 | rs1061170 |
| 9 | rs10801555 |
| 10 | rs2019727 |
| 11 | rs2019724 |
| 12 | rs203685 |
| 13 | rs1831281 |
| 14 | rs2274700 |
| 15 | rs6677604 |
| 16 | rs3753396 |
| 17 | rs419137 |
| 18 | rs2284664 |
| 19 | rs1065489 |
| 20 | rs10801560 |
| 21 | rs460897 |
| 22 | rs432007 |
| 23 | rs438781 |
| 24 | rs408519 |
| 25 | rs6428372 |
| 26 | rs10922147 |
| 27 | rs1971579 |
| 28 | rs4085749 |
| 29 | rs10922152 |
| 30 | rs5998 |

or at least one antibody with binding specificity to polypeptides encoded by any one polynucleotide sequence that comprises one or more polymorphisms selected from the list:

| SNP | |
|---|---|
| Number | SNP Name |
| 1 | rs1292487 |
| 2 | rs512900 |
| 3 | rs7524776 |
| 4 | rs529825 |
| 5 | rs800292 |
| 6 | rs1329424 |
| 7 | rs1061147 |
| 8 | rs1061170 |
| 9 | rs10801555 |
| 10 | rs2019727 |
| 11 | rs2019724 |
| 12 | rs203685 |
| 13 | rs1831281 |
| 14 | rs2274700 |
| 15 | rs6677604 |
| 16 | rs3753396 |
| 17 | rs419137 |
| 18 | rs2284664 |
| 19 | rs1065489 |
| 20 | rs10801560 |
| 21 | rs460897 |
| 22 | rs432007 |
| 23 | rs438781 |
| 24 | rs408519 |
| 25 | rs6428372 |
| 26 | rs10922147 |
| 27 | rs1971579 |
| 28 | rs4085749 |
| 29 | rs10922152 |
| 30 | rs5998 |

Suitably the array comprises at least one polypeptide encoded by any one polynucleotide sequence that comprises one or more polymorphisms selected from the list:
5 rs800292
8 rs1061170
15 rs6677604
16 rs3753396
17 rs419137
18 rs2284664
   or at least one antibody with binding specificity to polypeptides encoded by any one polynucleotide sequence that comprises one or more polymorphisms selected from the list:
5 rs800292
8 rs1061170
15 rs6677604
16 rs3753396
17 rs419137
18 rs2284664

As used herein, an antibody is defined in terms consistent in the art and includes monoclonal antibodies, polyclonal antibodies, fragments of such antibodies, including, but not limited to, Fab, F(ab′) and Fv fragments.

Many methods are known for generating and/or identifying antibodies to a known target peptide. The person of skill in the art would appreciate that existing techniques, for example the provision of isolated peptide to a mammalian organism including a rabbit, rat or mouse, to generate an immune response, could readily be used to provide suitable antibodies. As would be understood by those in the art, monoclonal antibodies can be produced by hybridomas. Hybridomas are immortalised cell lines, which can be created in vitro using two different cell types one of which is a tumour cell to create a cell capable of secreting a specific monoclonal antibody.

Diagnostic and assay means of detecting the presence of polypeptides or an immune responses to said polypeptides are known in the art. For example, the presence of the polypeptides may be detected by use of antibodies specific to said polypeptides.

Techniques which may be employed include, but are not limited to ELISA, Immunohistochemistry, Electron Microscopy, Latex agglutination, Immuno Blotting, immunochromatography, Immunochips, lateral flow immunoassays and Dip Stick immuno testing.

The ELISA test (enzyme linked immunoenzymatic assay) is frequently used for serological diagnosis. This method allows the identification and quantification of antigens or antibodies in biological fluids. The conventional ELISA consists in the detection of the complex antibody-antigen by a second antibody (against the antibody that reacts with the antigen) conjugated to an enzymatic activity (peroxidase, alkaline phosphatase and others).

In the latex agglutination assay, the antigen preparation is affixed to latex beads. The biological sample is then incubated directly on a slide with the latex particles. In a short time the reaction is examined for the presence of cross-linked or agglutinated latex particles indicating the presence of antibodies to polypeptides in the sample.

Immunochips may be used to determine the presence of the specific genetic markers of the invention. Generally, the specific antibodies to the antigens are immobilised on a transducer, e.g. electrodes, caloric meter, piezoelectric crystal, surface plasmon resonance transducer, surface acoustic resonance transducer or other light detecting device. The binding of antigens in the biological sample to the immobilised specific antibody is detected by a change in electrical signal.

The presence of the immunogenic antigens may be detected by detecting nucleic acids encoding the antigen or encoding antibodies raised against the antigen. Such techniques are well known in the art.

The determination by the inventor of polymorphisms which are associated to AMD can also be utilised in the diagnosis of AMD in a subject.

Accordingly, a further aspect of the invention, provides a method for the diagnosis of or predicting susceptibility to age-related macular degeneration in a subject, the method comprising the steps:

providing a biological sample from said subject;
determining the presence or absence of at least one genetic marker in the biological sample wherein said genetic marker is selected from a polynucleotide sequence that comprises one or more polymorphisms selected from the list:

| SNP | |
|---|---|
| Number | SNP Name |
| 1 | rs1292487 |
| 2 | rs512900 |
| 3 | rs7524776 |
| 4 | rs529825 |
| 5 | rs800292 |
| 6 | rs1329424 |
| 7 | rs1061147 |
| 8 | rs1061170 |
| 9 | rs10801555 |
| 10 | rs2019727 |
| 11 | rs2019724 |
| 12 | rs203685 |
| 13 | rs1831281 |
| 14 | rs2274700 |
| 15 | rs6677604 |
| 16 | rs3753396 |
| 17 | rs419137 |
| 18 | rs2284664 |
| 19 | rs1065489 |
| 20 | rs10801560 |
| 21 | rs460897 |
| 22 | rs432007 |
| 23 | rs438781 |
| 24 | rs408519 |
| 25 | rs6428372 |
| 26 | rs10922147 |
| 27 | rs1971579 |
| 28 | rs4085749 |
| 29 | rs10922152 |
| 30 | rs5998 |

or to a polypeptide encoded by at least one of said polynucleotide sequences,
wherein the presence and/or absence of a genetic marker is indicative of the risk of the subject developing Age Related Macular Degeneration (AMD).

Suitably the genetic markers are detected using the above identified polymorphisims.

Suitably said genetic marker is selected from a polynucleotide sequence that comprises one or more polymorphisms selected from the list:
5 rs800292
8 rs1061170
15 rs6677604
16 rs3753396
17 rs419137
18 rs2284664

Prediction of the onset of the disease in a subject permits early intervention and disease management, for example the provision of patient support services such as counselling. Early detection of the disease therefore enables patient treatment and management at an early stage.

The invention provides a method which can be used to determine the onset of AMD. The method can be used prior to the appearance of symptoms commonly used in the diagnosis of age-related macular degeneration. Thus, in preferred embodiments of the invention, the biological sample can be provided from a subject with no physical symptoms of AMD.

Any suitable biological sample can be used in the methods of the present invention. For example, the biological sample may be selected from the group comprising, but not limited to, biological fluid, such as sputum, saliva, plasma, blood, urine or a tissue, such as a biopsy of a tissue.

In the methods of the invention, the inventor considers that by testing for the presence of a plurality of genetic markers, for example, at least two genetic markers, at least three genetic markers, at least four genetic markers, at least five genetic markers, at least six genetic markers, at least ten genetic markers, at least fifteen genetic markers, the sensitivity of the method of diagnosis or prediction of onset of disease is improved.

In preferred embodiments of the method, the method comprises the steps:
providing a biological sample from a subject;
   determining the presence or absence of two or more genetic markers in the biological sample wherein the genetic markers are selected from a polynucleotide sequence that comprises one or more polymorphisms selected from the list:

| SNP | |
|---|---|
| Number | SNP Name |
| 1 | rs1292487 |
| 2 | rs512900 |
| 3 | rs7524776 |
| 4 | rs529825 |
| 5 | rs800292 |
| 6 | rs1329424 |
| 7 | rs1061147 |
| 8 | rs1061170 |
| 9 | rs10801555 |
| 10 | rs2019727 |
| 11 | rs2019724 |
| 12 | rs203685 |
| 13 | rs1831281 |
| 14 | rs2274700 |
| 15 | rs6677604 |
| 16 | rs3753396 |
| 17 | rs419137 |
| 18 | rs2284664 |
| 19 | rs1065489 |
| 20 | rs10801560 |
| 21 | rs460897 |
| 22 | rs432007 |
| 23 | rs438781 |
| 24 | rs408519 |
| 25 | rs6428372 |
| 26 | rs10922147 |
| 27 | rs1971579 |
| 28 | rs4085749 |
| 29 | rs10922152 |
| 30 | rs5998 |

or to a polypeptide encoded by at least one of said polynucleotide sequences,
wherein the presence and/or absence of a genetic marker is indicative of the risk of the subject developing Age Related Macular Degeneration (AMD).

Suitably the genetic markers are selected from a polynucleotide sequence that comprises one or more polymorphisms selected from the list:
5 rs800292
8 rs1061170
15 rs6677604
16 rs3753396
17 rs419137
18 rs2284664

According to a further aspect of the invention, there is provided a method of monitoring the progression of age-related macular degeneration from a first time-point to a later time-point, said method comprising the steps:

providing a first biological sample obtained at the first time-point,
determining the presence or absence of at least one genetic marker in said biological sample, wherein said genetic marker is selected from a polynucleotide sequence that comprises one or more polymorphisms selected from the list:

| SNP | |
|---|---|
| Number | SNP Name |
| 1 | rs1292487 |
| 2 | rs512900 |
| 3 | rs7524776 |
| 4 | rs529825 |
| 5 | rs800292 |
| 6 | rs1329424 |
| 7 | rs1061147 |
| 8 | rs1061170 |
| 9 | rs10801555 |
| 10 | rs2019727 |
| 11 | rs2019724 |
| 12 | rs203685 |
| 13 | rs1831281 |
| 14 | rs2274700 |
| 15 | rs6677604 |
| 16 | rs3753396 |
| 17 | rs419137 |
| 18 | rs2284664 |
| 19 | rs1065489 |
| 20 | rs10801560 |
| 21 | rs460897 |
| 22 | rs432007 |
| 23 | rs438781 |
| 24 | rs408519 |
| 25 | rs6428372 |
| 26 | rs10922147 |
| 27 | rs1971579 |
| 28 | rs4085749 |
| 29 | rs10922152 |
| 30 | rs5998 |

or to a polypeptide encoded by at least one of said polynucleotide sequences,
providing a second biological sample obtained at a later time-point,
determining the presence or absence of at least one genetic marker in said second biological sample, wherein said genetic marker is selected from a polynucleotide sequence that comprises one or more polymorphisms selected from the list:

| SNP | |
|---|---|
| Number | SNP Name |
| 1 | rs1292487 |
| 2 | rs512900 |
| 3 | rs7524776 |
| 4 | rs529825 |
| 5 | rs800292 |
| 6 | rs1329424 |
| 7 | rs1061147 |
| 8 | rs1061170 |
| 9 | rs10801555 |
| 10 | rs2019727 |
| 11 | rs2019724 |
| 12 | rs203685 |
| 13 | rs1831281 |
| 14 | rs2274700 |
| 15 | rs6677604 |
| 16 | rs3753396 |
| 17 | rs419137 |
| 18 | rs2284664 |
| 19 | rs1065489 |
| 20 | rs10801560 |
| 21 | rs460897 |
| 22 | rs432007 |
| 23 | rs438781 |
| 24 | rs408519 |
| 25 | rs6428372 |
| 26 | rs10922147 |
| 27 | rs1971579 |
| 28 | rs4085749 |
| 29 | rs10922152 |
| 30 | rs5998 |

or to a polypeptide encoded by at least one of said polynucleotide sequences,
comparing the absence and/or presence of said genetic marker and/or polypeptide in the second sample in relation to the first sample;
wherein a difference in the presence and/or absence of a genetic marker and/or polypeptide in the first sample in relation to the second sample is indicative of a change in the risk of the subject in developing AMD.

Suitably the genetic markers are selected from a polynucleotide sequence that comprises one or more polymorphisms selected from the list:
5 rs800292
8 rs1061170
15 rs6677604
16 rs3753396
17 rs419137
18 rs2284664

In particular embodiments of the methods of the invention, the methods comprise determining the presence and/or absence of at least three genetic markers and/or polypeptides, at least four genetic markers and/or polypeptides, at least five genetic markers and/or polypeptides, at least six genetic markers and/or polypeptides, at least ten genetic markers and/or polypeptides, at least fifteen genetic markers and/or polypeptides in the first and second samples.

Suitably in certain embodiments of the method, a subject can be provided with a possible therapeutic agent in the period between provision of a first and second biological sample and the detection of a genetic marker/polypeptide of the respective biological sample can be correlated with data on the effectiveness and responsiveness of that subject to the possible therapeutic agent with respect to age-related macular degeneration.

Suitably this provides a method for screening and selecting therapeutic agents and also for identifying subjects likely to respond to a particular therapeutic agent. Suitably, the pharmocogenomic susceptibility of a subject can be assessed to provide details of genetic variations in response to a drug or abnormal actions to drugs.

Preferred features and embodiments of each aspect of the invention are as for each of the other aspects mutatis mutandis unless context demands otherwise.

An embodiment of the invention is illustrated by way of example only with reference to the following figures wherein;

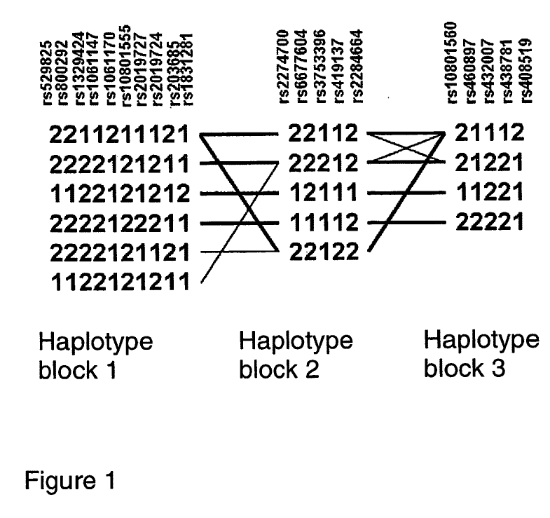

FIG. 1 shows the block structure and relationship between haplotypes in CFH.

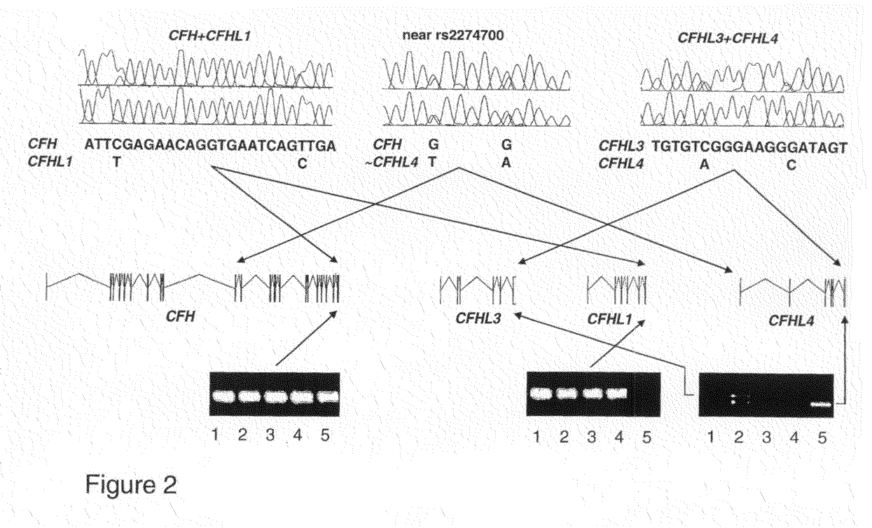

FIG. 2 shows the organisation of CFH, CFHL3, CFHL1 and CFHL4. Sequences show analysis of haplotype 5 (lower trace) and a representative of all other haplotypes (upper trace) of products co-amplifying from two loci. PCR products below the genes show amplification (using gene-specific primers) of CFH and CFHL4, but not of CFHL3 or CFHL1, in haplotype 5.

The present inventor has genotyped polymorphisms spanning the cluster of CFH and five CFH-like genes on chromosome 1q23 in 172 cases with severe neovascular AMD and 173 elderly controls with no signs of AMD. Detailed analysis of all haplotypes revealed a common deletion of CFHL1 and CFHL3 in 20% of chromosomes of controls that was strongly protective against development of AMD and of greater significance than Y402H.

Patients for the present study were recruited from opthalmology clinics and had choroidal neovascularization associated with the more severe exudative or wet form of AMD. The age-matched controls had no signs of age-related macular disease. The inventor typed 24 SNPs in the CFH region, including most common cSNPs, and additional intronic and intergenic SNPs selected to ascertain full haplotype information. Data confirmed the strong association between CFH and AMD²⁻⁵, which was evident both assessing SNPs individually and by haplotype (Table 1a,b). There was extensive linkage disequilibrium (LD) throughout the region. With the exception of rs1065489, all SNPs typed in CFH were placed within three large haplotype blocks spanning 45, 19 and 84 kb, respectively (FIG. 1). Block 3 markers extended from intron 21 of CFH to CFHL1. Genotyping allowed discrimination of all haplotypes of frequency greater than 1% found in final HapMap data⁶. The SNPs in block 3 returned significantly lower Illumina genotyping quality scores, reflecting poorer clustering of samples of identical genotype possibly influenced by the repetitive nature of this region, however, genotypes at these markers fell within Hardy Weinberg equilibrium, showed full LD within their haplotype block, and strong LD with haplotypes of neighboring block 2.

Within each block, the haplotypes ranged in effect from strongly detrimental to highly protective of AMD. In block 1, rs1061170 (Y402H) characterized one of two haplotypes associated with increased risk of AMD. Of greater significance to AMD status, however, was a strongly protective haplotype which showed a near-solid spine of LD across all blocks. This common haplotype was found in 20% of chromosomes of controls and conferred marginally its best protective odds ratio of 3.06 in block 3. It could be tagged uniquely by the C allele of rs460897 in block 3 or the A allele of rs6677604 in block 2. Individually, rs2274700 within haplotype block 2 was the best single predictor of case status (p=1.68×10⁻⁹). Both the G and A alleles of rs2274700 encode alanine at codon 473 and the polymorphism is not thought to have any functional role affecting splicing, however, it discriminated optimally between the groups of adverse and beneficial haplotypes.

The inventor aimed to identify the genetic basis of the strongly protective haplotype 5 (block 2:11112; block 3:22221). Of interest was the apparently reliable typing in the study of rs460897, a previously validated non-synonymous SNP reported to encode c.3572C>T (S1191 L) within the final exon of CFH. The reference coding sequence of this exon shares 99% homology with the final exon of CFHL1, differing only at c.3572 and c.3590, Genotyping of rs460897 was likely to account for the contribution of alleles at both exon 23 of CFH and exon 6 of CFHL1, with either deletion or conversion generating typing outcome. Gene-specific primers were selected to sequence these exons in DNA from homozygous individuals for each of the five haplotypes in block 2 and also in the associated haplotype in block 3. They showed no variation in exon 23 of CFH in association with any haplotype. Exon 6 of CFHL1 failed to amplify in homozygotes for the protective haplotype 5 (FIG. 2). In all other haplotypes, axon 6 of CFHL1 differed from CFH at the expected sites and also showed haplotype-specific variation at SNPs rs4320 (c.906G>T), and rs414628 (c.942A>T). All 28 samples sequenced from heterozygous individuals carrying one copy of haplotype 5 appeared to be homozygous at all CFHL1 axon 6 SNP sites, including 10 for a rare allele. Absence of any heterozygosity provided strong support for deletion of CFHL1 exon 6 on haplotype 5. Deletion was confirmed in haplotype 5 homozygotes by co-amplification using primers that annealed to sites common in both CFH and CFHL1. Sequencing of PCR products showed that haplotype 5 amplified only CFH, whereas all other haplotypes amplified both genes, revealing pseudoSNPs at sites differing between CFH and CFHL1 (FIG. 2). Deletion of CFHL1 intron 4 was similarly shown by co-amplification using primers common to CFH intron 21 and CFHL1 intron 4 which amplified products of 324 and 380 bp, respectively (FIG. 2). CFHL3 was also deleted from haplotype 5, as indicated by amplification only of CFHL4 sequence in homozygotes using primers specific to both exon 6 of CFHL3 and CFHL4 flanking a region of incomplete homology (FIG. 2). The exact position and size of the deletion has not been measured, however, is anticipated at about 80 kb based on the interval between two large segments of duplication in the physical map of the chromosome⁷. It does not extend as far as CFHL4, where an imperfect copy of CFH exon 11 sequence surrounding rs2274700 is inserted and retained on all haplotypes (FIG. 2). This duplication did not interfere with typing of rs2274700 using a reverse primer for extension. SNP rs1410996 located only in CFH intron 15 was in absolute LD with rs2274700 (data not shown).

Building the genome assembly in a region of extremely complex duplication is not straightforward. Sequence data on which the physical map is based was reviewed and agrees with the arrangement of CFH and related genes. An alternative Celera build in which the terminal exons of CFH are allelic with CFHL1, with a similar relationship between CFHL3 and CFHL4, is not supported by the present data, nor is gene conversion of CFHL1 from CFH. The inventor used multiplex ligation-dependent probe amplification (MLPA)⁸ to measure copy number of CFH exon 23 and CFHL1 exon 6. Assays centered on CFH c.3572C/CFHL1 c.869T and the hybridizing portions of the gene-specific probes varied only at one key base. The copy number of CFH exon 23 remained constant (1.00/1.04/1.06) in male and female DNA samples from individuals carrying 0, 1 or 2 copies of haplotype 5, when referenced to an autosomal marker in exon 9 of MORF4L1 and an X-linked marker in exon 6 of BCAP31 which was corrected for sex in males. As expected, the copy number of CFHL1 dropped from 1 to 0.44 and 0 in heterozygotes and homozygotes for haplotype 5, respectively.

CFH and CFHL1 are more important regulators of complement activity and are expressed at higher levels than the other CFH related proteins, hence it can be assumed that deletion of CFHL1 may be more significant than CFHL3 in protection against AMD. CFH and CFHL1 are present in the circulation at high levels and both act as co-factors for factor I-mediated degradation of C3b^{9,10}. Some insight about how CFHL1 deletion may protect against AMD comes from study of mutations in CFH which cause hemolytic uremic syndrome⁷ (HUS; OMIM #235400). Over 75% of known HUS mutations are clustered in the exons of CFH which share homology with CFHL1¹¹⁻¹³. Mutations in earlier exons tend to affect CFH protein stability in the plasma rather than function. Within exon 23, c.3572C>T (S1191 L) and c.3590T>C (V1197A) are found either separately or together. It is feasible that the HUS patients with both mutations may have a deletion similar to the one that protects against AMD but with earlier break points. This would result in conversion of CFH with CFHL1 and removal of CFHL3, instead of removal of CFHL3 and CFHL1 that protects against AMD. These HUS mutations cause decreased C3b binding and reduced ability to control complement activation on cellular surfaces⁷. The effect of substituting the final CFH exon with that of CFHL1 in HUS patients results in microanglopathic renal disease with parallels in our severe AMD patients who suffer from neovascular bleeding in the retina. The CFH gene cluster is responsible for numerous alternatively spliced transcripts and proteins. The final exon of CFHL1 may be alternatively spliced into CFH, and exons of CFHL3 and CFHL1 may participate in additional transcripts. Much work is required to unravel the complexity of these genes at the DNA level, and of the transcripts and proteins arising from this highly duplicated gene cluster. Other deletions or rearrangements can be anticipated. Overall, the data at present support a model in which CFH is required to maintain healthy microvasculature and CFHL1 is best viewed as a deleterious interfering fragment. The prevalence of age-related macular degeneration is growing in parallel with the increasing longevity of the population. With no effective treatment, AMD presents a major challenge. Starting to resolve the complex role of CFH and related genes in predisposition to AMD may shed some light on its etiology and in the future present useful therapeutic targets for gene silencing.

### Methods

### DNA Extraction, Genotyping and Sequencing

All participants were recruited in Northern Ireland, UK and were of Caucasian origin. DNA was extracted from peripheral blood by standard methods. High throughput SNP genotyping was outsourced using Illumina bead technology based on multiplex PCR and primer extension (Illumina, San Diego, USA) as part of a larger project. Additional SNPs were typed in-house using multiplex PCR followed by multiplex SNaPshot (ABI) technology. Primers were designed using Primer Detective (Clontech). Primer sequences for specific and non-specific amplification of CFH and related genes are available online. Sequencing was performed using ABI dye terminator chemistry v3 with analysis on an ABI3100 genetic analyzer. We used Sequencher program (Genecodes) to compare DNA sequences. SNP genotypes were numbered with the A or T allele designated 1 and the C or G allele designated 2. The A allele in the forward orientation was designated 1 in the two A/T SNPs rs2019727 and rs 438781.

### Statistical Methods

Genotype data were loaded into Haploview¹⁴ (www.broad.mit.edu/mpg/haploview/) in linkage format to generate case and control allele and haplotype numbers and ratios, and P values based on the chi-squared test for association of allele or haplotype frequencies. Data from 172 cases and 173 controls were used in our case:control study.

### MLPA

This was performed on 100 ng of DNA using buffers, enzymes and PCR primers from MRC Holland according to their protocol. Hybridizing probe sequences X-CFH-1191C or X-CFHL1-1191T were used in separate reactions with a common PHO-labelled oligo CFH1191-Y. Controls using MORF4L 1 and BCAP31 were included in all reactions. Analysis was based on peak heights, and correlated well with peak areas. All MLPA oligos are available online.

### GenBank Accession Numbers

CFH NM000186; CFHL1 BC016755; CFHL2 BC022283; CFHL3 AK124051; CFHL4 BC074957. CFH exons are numbered to include exon 10 which is alternatively spliced into the shorter transcript of this gene. Numbering of transcripts starts from the initial ATG.

### REFERENCES

1. Mullins, R. F., Aptsiauri, N. & Hageman G. S. Structure and composition of drusen associated with glomerulonephritis: implications for the role of complement activation in drusen biogenesis. Eye 15, 390-395 (2001).
2. Klein, R. J. et al. Complement factor H polymorphism in age-related macular degeneration. Science 308, 385-389 (2005).
3. Edwards, A. O. et al. Complement factor H polymorphism and age-related macular degeneration. Science 308, 421-424 (2005).
4. Haines, J. L. et al. Complement factor H variant increases the risk of age-related macular degeneration. Science 308, 419-421 (2005).
5. Hageman, G. S. et al. A common haplotype in the complement regulatory gene factor H (HF1/CFH) predisposes individuals to age-related macular degeneration. Proc. Natl. Acad. Sci. USA 102, 7227-7232 (2005).
6. The International HapMap Consortium. A haplotype map of the human genome. Nature 437, 1299-1320 (2005).
7. Heinen S et al. De novo gene conversion in the RCA gene cluster (1q32) causes mutations in complement factor H associated with atypical hemolytic uremic syndrome. Hum. Mut. In press.
8. Schouten, J. P. et al. Relative quantification of 40 nucleic acid sequences by multiplex ligation-dependent probe amplification. Nucleic Acids Res. 30, e57 (2002).
9. Reid, K. B. et al Complement system proteins which interact with C3b or C4b; a superfamily of structurally related proteins. Immunol. Today 7, 230-234 (1986).
10. DiScipio R G. Ultrastructures and interactions of complement factors H and I. J. Immunol.; 149, 2592-2599 (1992).
11. Warwicker, P. et al. Genetic studies into inherited and sporadic hemolytic uremic syndrome. Kidney Int. 53, 836-844, (1998).
12. Perez-Caballero, D. et al. Clustering of missense mutations in the C-terminal region of factor H in atypical hemolytic uremic syndrome. Am. J. Hum. Genet. 68, 478-484. (2001).
13. www.FH-HUS.org
14. Barrett, J. C., Fry, B., Maller, J. & Daly, M. J. Haploview: analysis and visualization of LD and haplotype maps. Bioinformatics. 21, 263-265 (2005).

TABLE 1a

| SNP | | | | | |
|---|---|---|---|---|---|
| Number | SNP Name | Coding Variant | Case, Control Ratios | Chi square | P value |
| 1 | rs1292487 | | 312:38, 263:77 | 17.3 | 3.26 × 10⁻⁵ |
| 2 | rs512900 | | 348:2, 338:2 | 0.0 | 0.98 |
| 3 | rs7524776 | | 322:28, 292:48 | 6.6 | 0.01 |
| 4 | rs529825 | | 313:37, 263:77 | 18.2 | 1.95 × 10⁻⁵ |
| 5 | rs800292 | CFHI62V | 313:37, 263:77 | 18.2 | 1.95 × 10⁻⁵ |
| 6 | rs1329424 | | 193:157, 130:210 | 19.8 | 8.59 × 10⁻⁶ |
| 7 | rs1061147 | CFHA307A | 193:157, 130:210 | 19.8 | 8.59 × 10⁻⁶ |
| 8 | rs1061170 | CFHY402H | 194:156, 130:210 | 20.5 | 6.06 × 10⁻⁶ |
| 9 | rs10801555 | | 194:156, 130:210 | 20.5 | 6.06 × 10⁻⁶ |
| 10 | rs2019727 | | 307:27, 272:66 | 18.4 | 1.75 × 10⁻⁵ |
| 11 | rs2019724 | | 216:134, 141:199 | 28.3 | 1.04 × 10⁻⁷ |
| 12 | rs203685 | | 215:135, 141:199 | 27.5 | 1.57 × 10⁻⁷ |
| 13 | rs1831281 | | 297:37, 269:69 | 11.0 | 0.0009 |
| 14 | rs2274700 | CFHA473A | 284:66, 205:135 | 36.3 | 1.68 × 10⁻⁹ |
| 15 | rs6677604 | | 323:27, 274:66 | 20.2 | 6.85 × 10⁻⁶ |
| 16 | rs3753396 | CFHQ872Q | 282:68, 278:62 | 0.2 | 0.69 |
| 17 | rs419137 | | 285:65, 296:44 | 4.1 | 0.043 |
| 18 | rs2284664 | | 311:39, 271:69 | 10.9 | 0.0009 |
| 19 | rs1065489 | CFHD936E | 281:69, 276:64 | 0.1 | 0.77 |
| 20 | rs10801560 | | 311:39, 274:66 | 9.14 | 0.0025 |
| 21 | rs460897 | CFHL1191S | 323:27, 270:68 | 22.2 | 2.42 × 10⁻⁶ |
| 22 | rs432007 | | 214:134, 145:191 | 23.1 | 1.57 × 10⁻⁶ |
| 23 | rs438781 | | 217:133, 148:192 | 23.6 | 1.18 × 10⁻⁶ |
| 24 | rs408519 | | 215:135, 147:193 | 22.9 | 1.72 × 10⁻⁶ |
| 25 | rs6428372 | | 285:65, 281:59 | 0.2 | 0.68 |
| 26 | rs10922147 | | 300:48, 261:79 | 10.2 | 0.0014 |
| 27 | rs1971579 | | 265:85, 223:117 | 8.5 | 0.0035 |
| 28 | rs4085749 | CFHL2C140C | 302:48, 263:77 | 9.3 | 0.0023 |

P values were generated using a chi-squared test for association of allele frequencies in 172 AMD patients and 173 controls.

TABLE 1b
Association of CFH gene haplotype blocks

| | Haplotype | % in controls | % in cases | Odds ratio | Chi Square | P Value |
|---|---|---|---|---|---|---|
| Block 1 | | | | | | |
| markers 4-13 | 2211211122 | 38.2 | 54.9 | −1.98 | 19.2 | 1.2 × 10⁻⁵ |
| | 2222121212 | 16.3 | 19.3 | −1.23 | 1.1 | 0.29 |
| | 1122121211 | 20.3 | 10.3 | +2.21 | 13.2 | 0.0003 |
| | 2222122212 | 19.6 | 7.9 | +2.86 | 19.8 | 8.6 × 10⁻⁶ |
| | 2222121122 | 3.2 | 6.0 | rare | 3.0 | 0.081 |
| | 1122121212 | 2.4 | 0.3 | rare | 5.5 | 0.019 |
| Block 2: | | | | | | |
| markers 14-18 | | | | | | |
| haplotype 1 | 22122 | 12.9 | 18.6 | −1.53 | 4.1 | 0.043 |
| haplotype 2 | 22112 | 29.1 | 43.1 | −1.85 | 14.7 | 0.0001 |
| haptotype 3 | 22212 | 18.2 | 19.4 | −1.08 | 0.2 | 0.69 |
| haplotype 4 | 12111 | 20.3 | 11.1 | +2.03 | 10.9 | 0.0009 |
| haplotype 5 | 11112 | 19.4 | 7.7 | +2.88 | 20.2 | 6.8 × 10⁻⁶ |
| Block 3 | | | | | | |
| markers 20-24 | 21112 | 43.2 | 61.5 | −2.10 | 23.0 | 1.6 × 10⁻⁶ |
| | 21221 | 17.2 | 19.5 | −1.17 | 0.6 | 0.43 |
| | 11221 | 19.4 | 11.2 | +1.91 | 9.0 | 0.0028 |
| | 22221 | 19.8 | 7.5 | +3.06 | 22.4 | 2.2 × 10⁻⁶ |

A negative odds ratio indicates a deleterious haplotype, and a positive indicates a protective AMD haplotype.

1. A medicament for the prevention of and/or treatment for AMD, the medicament comprising at least one inhibitor to wholly or partly silence at least one of gene CFHL1 and/or gene CFHL3.

2. A medicament as claimed in claim 1, wherein the at least one inhibitor comprises RNAi.

3. A medicament as claimed in claim 1, wherein the at least one inhibitor is an antisense molecule which is complementary to mRNA of at least one of gene CFHL1 and gene CFHL3 such that the respective gene product is reduced.

4. A medicament as claimed in claim 1, wherein the at least one inhibitor is an antisense molecule which is complementary to mRNA of at least one of gene CFHL1 and gene CFHL3 under highly stringent conditions of hybridisation such that the respective gene product is reduced.

5. A medicament as claimed in claim 4, wherein the at least one inhibitor comprises a nucleotide sequence with at least 90% sequence identity to ttcaGctgattcacctgttctcAaat (SEQ ID NO 5).

6. A medicament as claimed in claim 4, wherein the at least one inhibitor comprises a nucleotide sequence comprising ttcaGctgattcacctgttctcAaat (SEQ ID NO 5).

7. (canceled)

8. (canceled)

9. A method of treating AMD comprising the step of providing at least one inhibitor to wholly or partly silence at least one of gene CFHL1 and/or gene CFHL3 to a patient in need thereof.

10. A method according to claim 9, further comprising the step of providing at least one of an anti-VEGF treatment and a drug which minimises the likelihood of a subject smoking.

11. A medicament of claim 1, further comprising at least one of an anti-VEGF treatment and a drug which minimises the likelihood of a subject smoking.

12. (canceled)

13. A probe comprising an isolated polynucleotide sequence that comprises one or more polymorphisms selected from the list:

| SNP | |
|---|---|
| Number | SNP Name |
| 1 | rs1292487 |
| 2 | rs512900 |
| 3 | rs7524776 |
| 4 | rs529825 |
| 5 | rs800292 |
| 6 | rs1329424 |
| 7 | rs1061147 |
| 8 | rs1061170 |
| 9 | rs10801555 |
| 10 | rs2019727 |
| 11 | rs2019724 |
| 12 | rs203685 |
| 13 | rs1831281 |
| 14 | rs2274700 |
| 15 | rs6677604 |
| 16 | rs3753396 |
| 17 | rs419137 |
| 18 | rs2284664 |
| 19 | rs1065489 |
| 20 | rs10801560 |
| 21 | rs460897 |
| 22 | rs432007 |
| 23 | rs438781 |
| 24 | rs408519 |
| 25 | rs6428372 |
| 26 | rs10922147 |
| 27 | rs1971579 |
| 28 | rs4085749 |
| 29 | rs10922152 |
| 30 | rs5998 |

14. A probe as claimed in claim 13 wherein an isolated polynucleotide comprises
5 rs800292
8 rs1061170
15 rs6677604
16 rs3753396
17 rs419137
18 rs2284664

15. A diagnostic kit for the diagnosis and/or monitoring of age related macular degeneration in a subject, said kit comprising: a detection reagent with binding specificity for a polynucleotide sequence comprising one or more polymorphisms selected from the list:

| SNP | |
|---|---|
| Number | SNP Name |
| 1 | rs1292487 |
| 2 | rs512900 |
| 3 | rs7524776 |
| 4 | rs529825 |
| 5 | rs800292 |
| 6 | rs1329424 |
| 7 | rs1061147 |
| 8 | rs1061170 |
| 9 | rs10801555 |
| 10 | rs2019727 |
| 11 | rs2019724 |
| 12 | rs203685 |
| 13 | rs1831281 |
| 14 | rs2274700 |
| 15 | rs6677604 |
| 16 | rs3753396 |
| 17 | rs419137 |
| 18 | rs2284664 |
| 19 | rs1065489 |
| 20 | rs10801560 |
| 21 | rs460897 |
| 22 | rs432007 |
| 23 | rs438781 |
| 24 | rs408519 |
| 25 | rs6428372 |
| 26 | rs10922147 |
| 27 | rs1971579 |
| 28 | rs4085749 |
| 29 | rs10922152 |
| 30 | rs5998 |

or to a polypeptide encoded by a polynucleotide sequence that comprises one or more polymorphisms selected from the list:

| SNP | |
|---|---|
| Number | SNP Name |
| 1 | rs1292487 |
| 2 | rs512900 |
| 3 | rs7524776 |
| 4 | rs529825 |
| 5 | rs800292 |
| 6 | rs1329424 |
| 7 | rs1061147 |
| 8 | rs1061170 |
| 9 | rs10801555 |
| 10 | rs2019727 |
| 11 | rs2019724 |
| 12 | rs203685 |
| 13 | rs1831281 |
| 14 | rs2274700 |
| 15 | rs6677604 |
| 16 | rs3753396 |
| 17 | rs419137 |
| 18 | rs2284664 |
| 19 | rs1065489 |
| 20 | rs10801560 |
| 21 | rs460897 |
| 22 | rs432007 |
| 23 | rs438781 |
| 24 | rs408519 |
| 25 | rs6428372 |
| 26 | rs10922147 |
| 27 | rs1971579 |
| 28 | rs4085749 |
| 29 | rs10922152 |
| 30 | rs5998 |

16. An array comprising at least two polynucleotide sequences capable of hybridizing to at least two genetic markers selected from polynucleotide sequence that comprise one or more polymorphisms selected from the list:

| SNP | |
|---|---|
| Number | SNP Name |
| 1 | rs1292487 |
| 2 | rs512900 |
| 3 | rs7524776 |
| 4 | rs529825 |
| 5 | rs800292 |
| 6 | rs1329424 |
| 7 | rs1061147 |
| 8 | rs1061170 |
| 9 | rs10801555 |
| 10 | rs2019727 |
| 11 | rs2019724 |
| 12 | rs203685 |
| 13 | rs1831281 |
| 14 | rs2274700 |
| 15 | rs6677604 |
| 16 | rs3753396 |
| 17 | rs419137 |
| 18 | rs2284664 |
| 19 | rs1065489 |
| 20 | rs10801560 |
| 21 | rs460897 |
| 22 | rs432007 |
| 23 | rs438781 |
| 24 | rs408519 |
| 25 | rs6428372 |
| 26 | rs10922147 |
| 27 | rs1971579 |
| 28 | rs4085749 |
| 29 | rs10922152 |
| 30 | rs5998 |

17. A polypeptide array, wherein said polypeptide array is comprised of polypeptides encoded by polynucleotide sequence that comprise one or more polymorphisms selected from the list:

| SNP | |
|---|---|
| Number | SNP Name |
| 1 | rs1292487 |
| 2 | rs512900 |
| 3 | rs7524776 |
| 4 | rs529825 |
| 5 | rs800292 |
| 6 | rs1329424 |
| 7 | rs1061147 |
| 8 | rs1061170 |
| 9 | rs10801555 |
| 10 | rs2019727 |
| 11 | rs2019724 |
| 12 | rs203685 |
| 13 | rs1831281 |
| 14 | rs2274700 |
| 15 | rs6677604 |
| 16 | rs3753396 |
| 17 | rs419137 |
| 18 | rs2284664 |
| 19 | rs1065489 |
| 20 | rs10801560 |
| 21 | rs460897 |
| 22 | rs432007 |
| 23 | rs438781 |
| 24 | rs408519 |
| 25 | rs6428372 |
| 26 | rs10922147 |
| 27 | rs1971579 |
| 28 | rs4085749 |
| 29 | rs10922152 |
| 30 | rs5998 |

or at least one antibody with binding specificity to polypeptides encoded by polynucleotide sequence that comprise one or more polymorphisms selected from the list:

| SNP | |
|---|---|
| Number | SNP Name |
| 1 | rs1292487 |
| 2 | rs512900 |
| 3 | rs7524776 |
| 4 | rs529825 |
| 5 | rs800292 |
| 6 | rs1329424 |
| 7 | rs1061147 |
| 8 | rs1061170 |
| 9 | rs10801555 |
| 10 | rs2019727 |
| 11 | rs2019724 |
| 12 | rs203685 |
| 13 | rs1831281 |
| 14 | rs2274700 |
| 15 | rs6677604 |
| 16 | rs3753396 |
| 17 | rs419137 |
| 18 | rs2284664 |
| 19 | rs1065489 |
| 20 | rs10801560 |
| 21 | rs460897 |
| 22 | rs432007 |
| 23 | rs438781 |
| 24 | rs408519 |
| 25 | rs6428372 |
| 26 | rs10922147 |
| 27 | rs1971579 |
| 28 | rs4085749 |
| 29 | rs10922152 |
| 30 | rs5998 |

18. A method for the diagnosis of or predicting susceptibility to age-related macular degeneration in a subject, the method comprising the steps:
providing a biological sample from said subject;
determining the presence or absence of at least one genetic marker in the biological sample wherein said genetic marker is selected from a polynucleotide sequence that comprises one or more polymorphisms selected from the list:

| SNP | |
|---|---|
| Number | SNP Name |
| 1 | rs1292487 |
| 2 | rs512900 |
| 3 | rs7524776 |
| 4 | rs529825 |
| 5 | rs800292 |
| 6 | rs1329424 |
| 7 | rs1061147 |
| 8 | rs1061170 |
| 9 | rs10801555 |
| 10 | rs2019727 |
| 11 | rs2019724 |
| 12 | rs203685 |
| 13 | rs1831281 |
| 14 | rs2274700 |
| 15 | rs6677604 |
| 16 | rs3753396 |
| 17 | rs419137 |
| 18 | rs2284664 |
| 19 | rs1065489 |
| 20 | rs10801560 |
| 21 | rs460897 |
| 22 | rs432007 |
| 23 | rs438781 |
| 24 | rs408519 |
| 25 | rs6428372 |
| 26 | rs10922147 |
| 27 | rs1971579 |
| 28 | rs4085749 |
| 29 | rs10922152 |
| 30 | rs5998 |

or to a polypeptide encoded by at least one said polynucleotide sequence,
wherein the presence and/or absence of a genetic marker is indicative of the risk of the subject developing Age Related Macular Degeneration (AMD).

19. A method of monitoring the progression of age-related macular degeneration from a first time-point to a later time-point, said method comprising the steps:
providing a first biological sample obtained at the first time-point,
determining the presence or absence of at least one genetic marker in said biological sample, wherein said genetic marker is selected from a polynucleotide sequence that comprises one or more polymorphisms selected from the list:

| SNP | |
|---|---|
| Number | SNP Name |
| 1 | rs1292487 |
| 2 | rs512900 |
| 3 | rs7524776 |
| 4 | rs529825 |
| 5 | rs800292 |
| 6 | rs1329424 |
| 7 | rs1061147 |
| 8 | rs1061170 |
| 9 | rs10801555 |
| 10 | rs2019727 |
| 11 | rs2019724 |
| 12 | rs203685 |
| 13 | rs1831281 |
| 14 | rs2274700 |
| 15 | rs6677604 |
| 16 | rs3753396 |
| 17 | rs419137 |
| 18 | rs2284664 |
| 19 | rs1065489 |
| 20 | rs10801560 |
| 21 | rs460897 |
| 22 | rs432007 |
| 23 | rs438781 |
| 24 | rs408519 |
| 25 | rs6428372 |
| 26 | rs10922147 |
| 27 | rs1971579 |
| 28 | rs4085749 |
| 29 | rs10922152 |
| 30 | rs5998 |

or to a polypeptide encoded by at least one said polynucleotide sequence,
providing a second biological sample obtained at a later time-point,
determining the presence or absence of at least one genetic marker in said second biological sample, wherein said genetic marker is selected from a polynucleotide sequence that comprises one or more polymorphisms selected from the list:

| SNP | |
|---|---|
| Number | SNP Name |
| 1 | rs1292487 |
| 2 | rs512900 |
| 3 | rs7524776 |
| 4 | rs529825 |
| 5 | rs800292 |
| 6 | rs1329424 |
| 7 | rs1061147 |
| 8 | rs1061170 |
| 9 | rs10801555 |
| 10 | rs2019727 |
| 11 | rs2019724 |
| 12 | rs203685 |
| 13 | rs1831281 |
| 14 | rs2274700 |
| 15 | rs6677604 |
| 16 | rs3753396 |
| 17 | rs419137 |
| 18 | rs2284664 |
| 19 | rs1065489 |
| 20 | rs10801560 |
| 21 | rs460897 |
| 22 | rs432007 |
| 23 | rs438781 |
| 24 | rs408519 |
| 25 | rs6428372 |
| 26 | rs10922147 |
| 27 | rs1971579 |
| 28 | rs4085749 |
| 29 | rs10922152 |
| 30 | rs5998 |

or to a polypeptide encoded by at least one said polynucleotide sequence,
comparing the absence and/or presence of said genetic marker and/or polypeptide in the second sample in relation to the first sample;
wherein a difference in the presence and/or absence of a genetic marker and/or polypeptide in the first sample in relation to the second sample is indicative of a change in the risk of the subject in developing AMD.
